(19) United States
(12) Patent Application Publication
(10) Pub. No.: US 2019/0000314 A1
(43) Pub. Date: Jan. 3, 2019

(54) Systems and Methods for Posterior Segment Visualization and Surgery

(71) Applicant: NANOPHTHALMOS LLC, Miami, FL (US)

(72) Inventor: Richard M. Awdeh, Miami Beach, FL (US)

(73) Assignee: NANOPHTHALMOS LLC, Miami, FL (US)

(21) Appl. No.: 16/101,679

(22) Filed: Aug. 13, 2018

Related U.S. Application Data
(63) Continuation of application No. PCT/US2017/017586, filed on Feb. 12, 2017.
(60) Provisional application No. 62/294,932, filed on Feb. 12, 2016.

(51) Int. Cl. A61B 3/10; A61B 5/00; A61B 34/20; A61B 90/00

(52) U.S. Cl. CPC A61B 3/102; A61B 5/0066; A61B 5/061; A61B 90/39; A61B 34/20

(57) ABSTRACT

Systems and processes for performing posterior segment visualization and surgery assist a surgeon during surgery. Adapted instruments (I) used in ocular surgery include unique indicia, markings, or the like (40) which, when in the surgical field, can be identified by the system (FIG. 9). In turn, the system modifies the images that are projected into a display (10), e.g., a heads-up display, used by the surgeon, to display parameter data that is particular to the instrument (I) in use. The system also tracks the tip (16) of the instrument (I) in real time, determines the tissue that is closest to the instrument tip, automatically collects OCT data for that tissue, and displays images (20, 22) representative of that data in the display used by the surgeon. In this way, as the surgeon moves a surgical instrument around in the surgical field of the eye, the system automatically displays OCT image data of the tissue closest to the instrument tip.

### BACKGROUND

### Field of Endeavor

The present invention relates to devices, systems, and processes useful in ocular surgery, and more specifically for posterior segment ocular surgery.

### Brief Description of the Related Art

Prior systems have been described and are commercially available to assist in ocular surgery, and have assisted surgeons in the visualization of surgical parameters that relate to the surgery, presented in conjunction with the images presented by a surgical microscope. These include, but are not limited to, those described in the following published documents, the entirety of each of which is incorporated by reference herein: U.S. Pat. No. 7,800,820; U.S. Patent App. Publ. Nos. 2012/0330129, 2013/0073310, 2013/0304236, and 2015/0077528; and International App. Nos. 2015/138988 A1 and 2015/138994 A2. Commercially, such systems are available as 3D guidance technologies for cataract and refractive surgeries inside ophthalmic microscopes, as the Cirle Surgical Navigation System. While the systems and methods described in these documents, and commercially available, have provided great advances in anterior ocular surgery, including cataract surgeries, there has remained a need for systems which can assist the surgeon with posterior segment ocular surgery. Surgeries which require access to, and visualization of, the posterior segment of the ocular globe, e.g., retina surgeries, are problematic because of a significantly reduced ability to visualize the tissues of the posterior segment of the ocular globe, among other difficulties. Poor visualization is often due to complex optics challenges within the posterior segment, and visual confirmation of successful surgery is limited by poor visualization, resulting in higher re-operation rates. Thus, surgical plans are limited by how well the surgeon can visualize the tissue.

OCT is a well-understood and commercially implemented medical imaging method that uses light to capture micrometer-resolution, three-dimensional images from within optical scattering media (e.g., biological tissue). Optical coherence tomography is based on low-coherence interferometry, typically employing near-infrared light. The use of relatively long wavelength light allows it to penetrate into the scattering medium. Confocal microscopy, another optical technique, typically penetrates less deeply into the sample but with higher resolution.

Depending on the properties of the light source (e.g., superluminescent diodes, ultrashort pulsed lasers, supercontinuum lasers), optical coherence tomography has achieved sub-micrometer resolution (with very wide-spectrum sources emitting over a ˜100 nm wavelength range). Optical coherence tomography is one of a class of optical tomographic techniques. A relatively recent implementation of optical coherence tomography, frequency-domain optical coherence tomography, provides advantages in signal-to-noise ratio, permitting faster signal acquisition. Commercially available optical coherence tomography systems are employed in diverse applications, including art conservation and diagnostic medicine, notably in ophthalmology and optometry where it can be used to obtain detailed images from within the retina.

Benefits of OCT include: live sub-surface images at near-microscopic resolution; instant, direct imaging of tissue morphology; no preparation of the sample or subject; and no ionizing radiation. OCT delivers high resolution because it is based on light, rather than sound or radio frequency. An optical beam is directed at the tissue, and a small portion of this light that reflects from sub-surface features is collected. Note that most light is not reflected but, rather, scatters off at large angles. In conventional imaging, this diffusely scattered light contributes background that obscures an image. However, in OCT, a technique called interferometry is used to record the optical path length of received photons allowing rejection of most photons that scatter multiple times before detection. Thus OCT can build up clear 3D images of thick samples by rejecting background signal while collecting light directly reflected from surfaces of interest.

### SUMMARY

According to a first aspect of the invention, a system useful in posterior segment visualization and surgery includes at least two vitrectomy instruments, each instrument including external surface indicia that is different from the other of said at least two vitrectomy instruments, an optical data gathering system configured and arranged for use during posterior segment visualization and surgery, an instrument-identification system in communication with the optical data gathering system, the instrument-identification system including data representative of said external surface indicia and data representative of a type of vitrectomy instrument associated with said external surface indicia.

The system can further comprise a display system in communication with said instrument-identification system, said dis-play system configured and arranged to display parameter data based on said data representative of a type of vitrectomy instrument associated with said external surface indicia.

The display system can be further configured and arranged to not display parameter data not associated with said data representative of a type of vitrectomy instrument associated with said external surface indicia.

The display can be a heads-up display.

The external surface indicia can be visually detectable.

The external surface indicia can be are adjacent to a distal tip of each instrument.

According to a second aspect of the present invention, a system useful in posterior segment visualization and surgery comprises an optical data gathering system configured and arranged for use during posterior segment visualization and surgery, an instrument-tracking system in communication with said optical data gathering system, an optical computer tomography (OCT) data gathering system configured for use during posterior segment visualization and surgery, a display system configured and arranged to display images representative of data from said OCT system, and a processing system configured and arranged to receive data from said instrument-tracking system indicative of a location of a tip of a vitrectomy instrument, to direct said OCT system to gather OCT data concerning tissue closest to said instrument tip, and to direct said display system to display said OCT data.

The display system of the second aspect can be configured and arranged to display said OCT data immediately adjacent to the location of said instrument tip.

In the system of the second aspect according to claim 8, said OCT system is configured and arranged to gather 2D OCT data in two orthogonal planes, and said display system is configured and arranged to display said 2D OCT data intersecting at a point immediately adjacent to said instrument tip.

According to a third aspect of the present invention, a method of operating a posterior segment visualization and surgery system comprises (a) locating the tip of a vitrectomy instrument with an optical data gathering system, (b) gathering data with an optical computer tomography (OCT) data gathering system concerning tissue closest to said instrument tip, (c) displaying an image representative of said OCT data; moving said instrument tip to a new location; and repeating steps (a), (b), and (c) for said new location of said instrument tip.

In the method, displaying an image can further comprise displaying an image representative of the instrument tip.

The method can further comprise identifying external surface indicia on said instrument with said optical data gathering system, and displaying an image representative of parameter data of the use of the instrument.

Still other aspects, features, and attendant advantages of the present invention will become apparent to those skilled in the art from a reading of the following detailed description of embodiments constructed in accordance therewith, taken in conjunction with the accompanying drawings.

### BRIEF DESCRIPTION OF THE DRAWINGS

The invention of the present application will now be described in more detail with reference to exemplary embodiments of the apparatus and method, given only by way of example, and with reference to the accompanying drawings, in which:

FIG. 1 illustrates an exemplary embodiment of a HUD image that is generated by the systems described herein, superimposed on a real-time microscope image of a patient's retina;

FIG. 2 illustrates a 3D HUD overlay version of the images of FIG. 1;

FIG. 3 illustrates an enlarged view of a 3D representation of OCT data of tissue layers;

FIG. 4. illustrates another exemplary embodiment, similar to that illustrated in

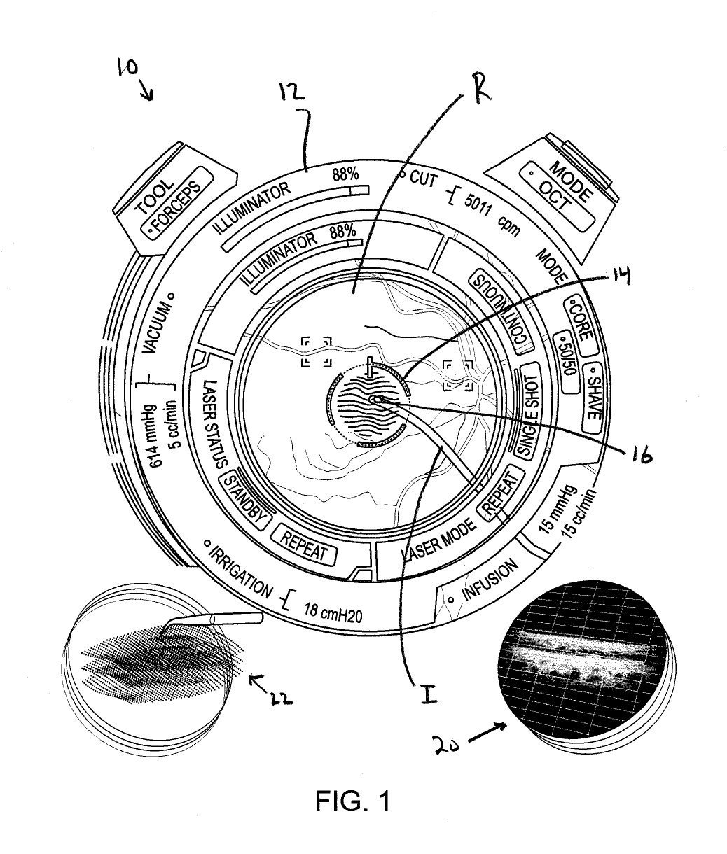

FIG. 1, including an X-Y OCT image overlayed at the instrument tip;

FIG. 5 illustrates a high-level method of performing posterior segment visualization and surgery;

FIG. 6 illustrates an exemplary method of identifying an instrument tip;

FIG. 7 illustrates an exemplary method of identifying an instrument type and modifying display data;

FIG. 8 illustrates an instrument including several optional features; and

FIG. 9 illustrates a system.

### DETAILED DESCRIPTION OF EXEMPLARY EMBODIMENTS

Referring to the drawing figures, like reference numerals designate identical or corresponding elements throughout the several figures.

The singular forms “a,” “an,” and “the” include plural referents unless the context clearly dictates otherwise. Thus, for example, reference to “a solvent” includes reference to one or more of such solvents, and reference to “the dispersant” includes reference to one or more of such dispersants.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

For example, a range of 1 to 5 should be interpreted to include not only the explicitly recited limits of 1 and 5, but also to include individual values such as 2, 2.7, 3.6, 4.2, and sub-ranges such as 1-2.5, 1.8-3.2, 2.6-4.9, etc. This interpretation should apply regardless of the breadth of the range or the characteristic being described, and also applies to open-ended ranges reciting only one end point, such as “greater than 25,” or “less than 10.”

In general terms, beginning with the systems described in the aforementioned published patent applications, and the Cirle system, as a platform, the inventors hereof have developed systems and methods which are particularly useful to assist the surgeon when performing surgeries in and on the posterior segment of the ocular globe. The value of posterior segment navigation improvements described herein include increases in surgeon confidence and surgical outcome by allowing confirmation of surgical goals, improvements in surgeon decision making through digital visualization enhancements, and improvements in the reproducibility of the surgery, by being able to see how surgical maneuvers change the micro-anatomy.

Like the Cirle SNS and the systems described in the aforementioned patent documents, the systems described herein present a 3D holographic heads up display (HUD) in the surgical microscope. Different from the Cirle SNS and the systems described in the aforementioned patent documents, the systems described herein present an entirely different set of holographic images, based on different data collected during vitrectomy surgery, and manipulate the data and the resulting rendered holographic images differently, to address the different needs of the surgeon when operating in the posterior segment ocular surgery, including surgery on the retina.

During vitrectomy, the surgical team performs a number of steps, including: insertion of trochars and instrumentation into the eye; insertion of a light pipe into the eye; performing a pars plana vitrectomy; and monitoring and manipulating vitreous parameters (see FIG. 5). When visualizing the correct tissue plane as part of the vitrectomy, the surgeon uses light from the light pipe, the microscope, and the biom to view the proper location of the retina; traditionally, the surgeon uses stains on the target tissue to try to differentiate and ascertain the different tissue planes. In the systems of this application, the HUD presents a 3D OCT representation of the target tissue, and advantageously also displays a 3D representation of the tissue based on the use of digital filters to show the different tissue planes and any modification of that tissue by the instruments being used in the surgery. More specifically, spectral filtering of the light being backscattered from the issue, which is being radiated with an OCT beam, is performed. Filtering for different wavelengths of this return signal can highlight a specific depth plane.

Guidance to the proper location of pathology in the eye is currently accomplished by using light from the light pipe, with the microscope, and the biom to view the proper location of retina, which is currently enhanced with 2D OCT, often including a foot pedal which controls the OCT system. Different from the prior systems, the systems described herein optionally, yet advantageously, include the presentation of images derived from OCT data which is guided by the location of the instrument in the ocular globe, which provides an OCT overlay in the HUD of the relevant surgical point of interest. More specifically, and with reference to FIG. 6, the real-time OCT image that is rendered is of the tissue closest to the tip of the instrument, by the system tracking the location of the instrument's tip and gathering OCT data of the tissue closest to the tip. Stated somewhat differently, instrument tracking and the presentation of corresponding OCT images are essentially simultaneous. Furthermore, by providing an OCT image which is instead automatically based on the location of the surgical instrument in the eye, the surgeon is better able to confirm the anatomical success of the procedure. Thus, in the exemplary process illustrated in FIG. 6, the instrument tip is positioned in the patient's eye; image data of the surgical field of the eye is obtained by the system; the tip of the instrument is identified from that image data, as well as the tissue which is closest to the tip and its location; the system collects OCT data as otherwise described herein; and OCT data images are output the display device, e.g., HUD. As the instrument tip is moved, the process loops back to obtaining new image data, and the process is repeated to update the real-time OCT data images for a new location of the instrument tip.

Identifying the instrument tip can be done any of numerous ways. In one such exemplary process, the system identifies the instrument, and then identifies where the instrument ends within the surgical field, and assigns that portion of the instrument as the tip. In another exemplary process, the identity of the instrument (e.g., hook) is either input into the system, or determined by a process described elsewhere herein, and the 3D shape of the instrument is looked up in a database in the system, or to which the system otherwise has access. Once the shape of the instrument is thus known to the system, the system can compare the image data to the known shape of the instrument to identify the location of the tip of the instrument. According to yet another exemplary process of tip identification, the instrument tip can include a visual marker or the like so that it has a unique appearance as compared to the remainder of the instrument, from which the system can identify the tip.

Currently, it is only visual clues that give surgeons a best guess as to instrument location, and there is no tactile feedback during vitrectomy surgery. In the systems described herein, the instruments themselves are optionally modified to be detectable by the system, so that their location within the eye can be accurately, and automatically, determined by the system, and images representative of that location information rendered and displayed in the HUD. Particularly, the distance of the instrument (e.g., its tip) to the surface of the retina is derived from the detected location of the instrument and the location of the retina, and that distance information is presented in the HUD.

FIG. 1 illustrates an exemplary embodiment of a HUD image 10 that is generated by the systems described herein, superimposed on a real-time microscope image of a patient's retina R. In the center of the image is the real-time microscope image of the surgical field, that is, the retina R, while the remainder of the image is a computer-generated 3D HUD image 12 which changes depending on the stage of the surgery, the operating parameters of the surgical instruments, and the OCT data that is generated, among other things. More specifically, the system generates a HUD image overlay that includes one or more of the parameters of the surgical procedure:

laser parameters (mode of repeat, single shot, or continuous)

cutting parameters (mode of core or shave, cut speed in cpm)

infusion parameters (pressure, rate)

irrigation (pressure)

vacuum (pressure, rate)

illumination (e.g., percent of maximum)

identification of the tool currently being used

OCT mode

Optionally, the HUD includes an overlay which includes a representation of a target, circle, brackets, or the like, 14, which is generated to overlay the actual location of a portion (preferably, the tip) of the instrument I in use, in the actual surgical microscope image field. Thus, when the light is poor in the particular location of the instrument tip 16, or its location is difficult to see in the ocular globe, the instrumentation of the system locates the (tip of the) instrument and presents a locator image over it, so the surgeon can be apprised of the instrument's location.

In addition to the foregoing, which can be conveniently rendered to encircle the circular view generated by the surgical microscope in one or more segmented rings, the HUD can include overlays of a 2D OCT image 20 tracked to the instrument's tip, and optionally a 3D representation of the tissue layers 22, which is derived from the OCT data, juxtaposed with the instrument's location.

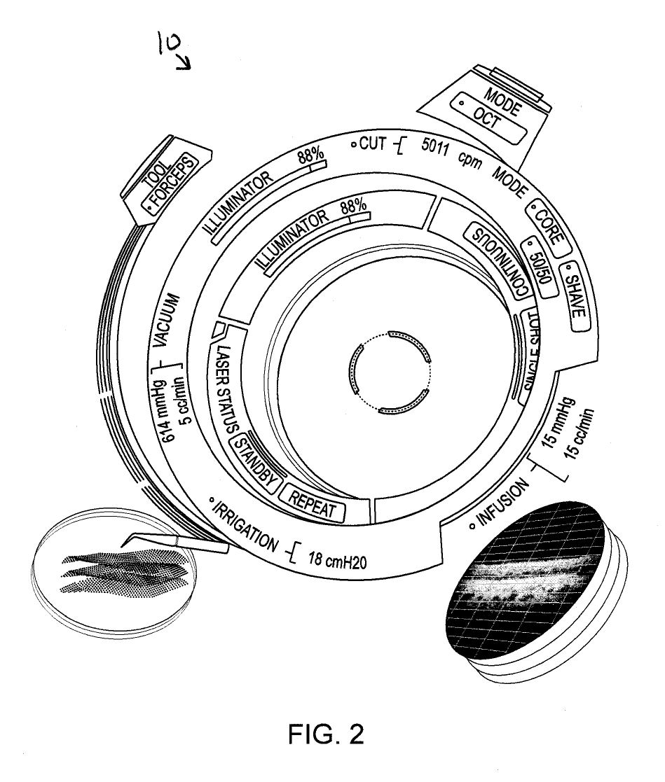

FIG. 2 illustrates a 3D HUD overlay version of the images of FIG. 1, demonstrating the depth of field that can be achieved using a 3D rendering engine to generate the image overlay data.

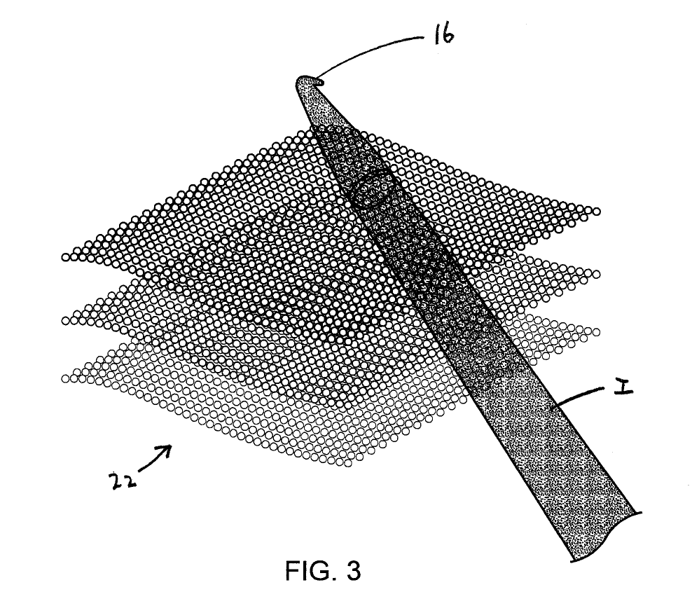

FIG. 3 illustrates an enlarged view of the 3D representation of the tissue layers 22, derived from the OCT data, juxtaposed with the instrument's location, of FIGS. 1 and 2. Because actual visualization of the tissue layers is extremely difficult, and current 2D OCT images only show an image of the tissue at a single plane into the tissue, the 3D representation can be particularly useful to the surgeon. By assembling the data from numerous 2D OCT scans taken in adjacent planes, the system can generate a data set representative of the tissue in three dimensions, generate a 3D model of the tissue layers from that data set, render a 3D image representative of that portion of the tissue, and can locate the instrument tip relative to the layers of the particular tissue of interest. While numerous methodologies can be employed, an example is described in “Real-time three-dimensional Fourier-domain optical coherence tomography video image guided microsurgeries”, Kang, Jin U., et al., Journal of Biomedical Optics, vol. 17(8), pp. 081403-1-6 (August 2012), the entirety of which is incorporated by reference herein.

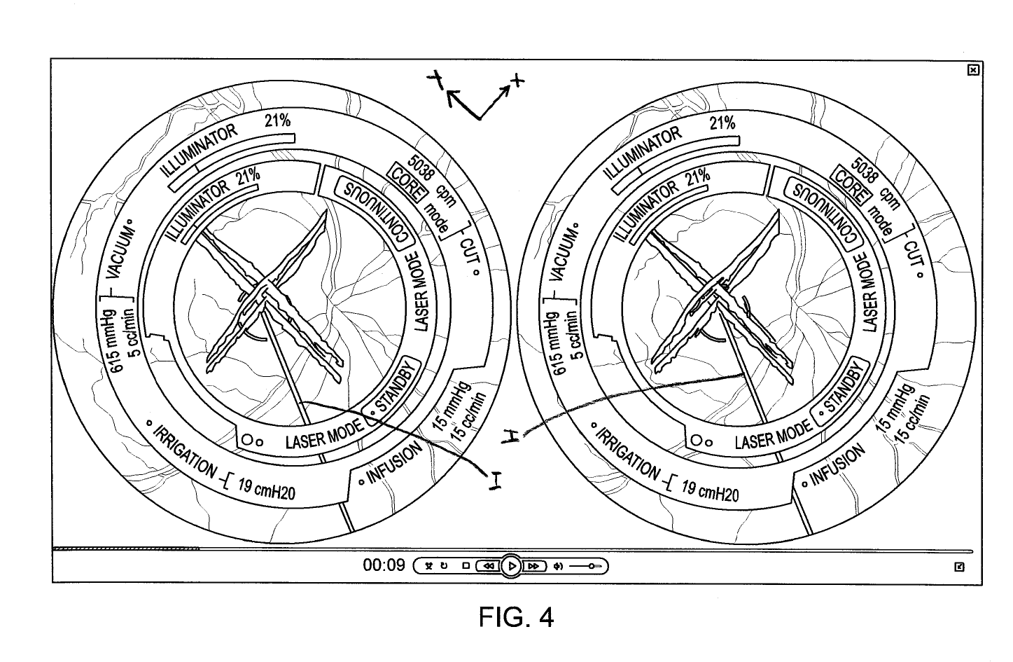

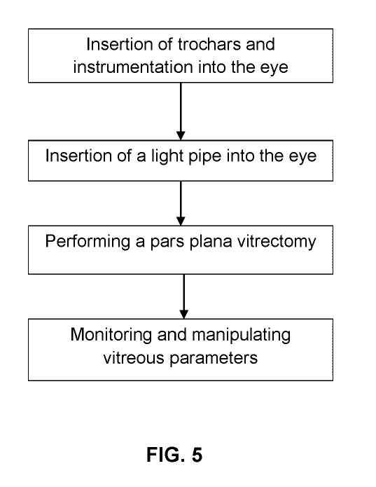

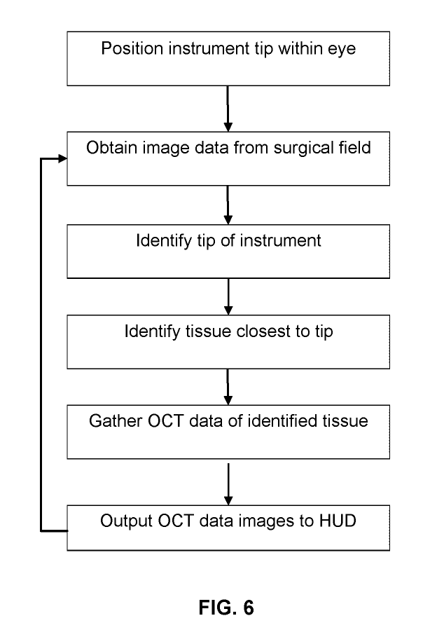

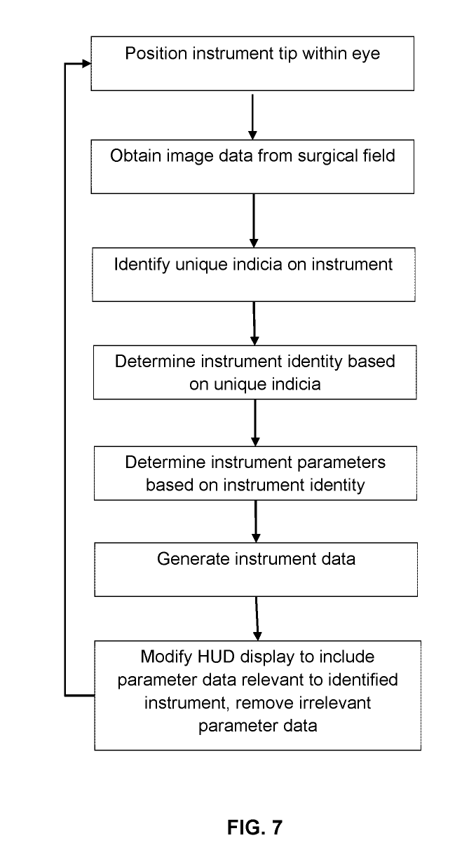

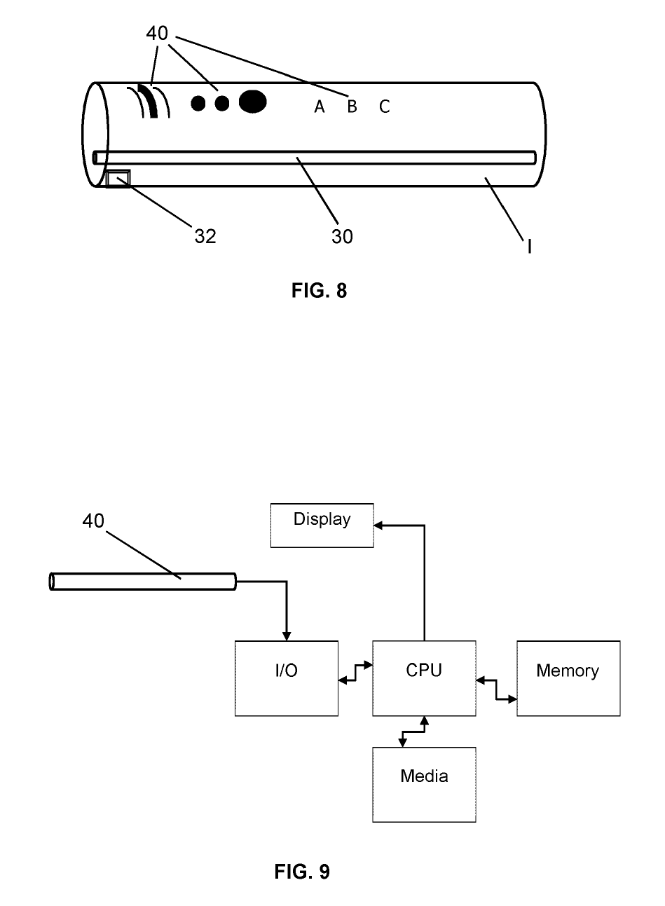

FIG. 4 illustrates yet another exemplary embodiment which is similar in many respects to that illustrated in FIG. 1. In the embodiment of FIG. 4, however, the OCT data is collected and rendered in the HUD in both the X and Y axes (the Z axis being that which extends perpendicular to surface of the closest tissue in the posterior segment); the two sets of perpendicular 2D OCT images extend in the X and Y directions. Further advantageously, the X-Y OCT dynamic image can be positioned at the tissue point at which the OCT data originates, thus providing to the surgeon this data immediately at the point of most interest and at which her focus is cast. In the illustrated embodiment, the instrument I is represented by the light line extending diagonally from the bottom right, with its tip being encircled by a broken line under the OCT images. Optionally, the X-Y OCT image can be rendered as a 3D cube instead of the pairs of 2D OCT images.

In other embodiments, the image overlays can be rendered in viewing devices other than the surgical microscope. By way of non-limiting example, some or all of the images described herein can be rendered in a head-directed display (such as those developed by Google), a virtual reality (VR) blocking display (such as those developed by Oculus, Inc.), a virtual reality overlay display (such as those developed by Magic Leap and Google), and/or a 3D television, including both those that do and do not require the 3D glasses.

In the systems described herein, adjacent to the image of the interior of the ocular globe that the surgeon sees through the surgical microscope, the HUD image includes at least one, and advantageously more than one, of the images described herein. These can include at least the vitrectomy parameters, e.g., cut speed and laser status, key indicators, and any warning indicators. The data for generating these images originates in the vitrectomy system used by the surgical team, but instead of being presented on external monitors and devices, the data is instead delivered to the processors and image generators such as those described in the Cirle SNS and the aforementioned patent documents, to be displayed in the HUD.

Embodiments of the systems and methods described herein use software filters on the OCT data to break up the data into discrete tissue layers, and then present a 3D holographic representation of those layers, optionally, yet advantageously, with either an actual image, or a holographic representation, of the surgical instrument that is being used by the surgeon to dissect, remove, or otherwise manipulate those layers of tissue in real time; this is represented in the abstract in FIG. 3, in which the small spheres are arranged in three representative layers under a representative instrument I having a tip 16. Additionally, current OCT data is presented in a two-dimensional format, i.e., a representation of a cross-sectional view of the tissue that is scanned by the OCT system, so the surgeon has representations in both 2D and 3D formats.

Embodiments of the systems and methods described herein also can use surgical instruments I which can interact with the rest of the system. More specifically, standard vitrectomy instruments (including, but not limited to, forceps, scissors, needleholders, retractors, hooks, picks, pneumatic cutting probe, irrigator probe, aspirator probe, spatula, laser probe, light pipe, membrane peeler, etc.) are modified so that the system described herein can identify and track the surgical instruments in real time, when inside the ocular globe. The system can then, based on the tracked location of the instrument, and more particularly the tip of the instrument, move the OCT system's area of data collection with the tracked motion of the surgical instrument(s) that are in the ocular globe, more specifically collecting data from the tissue closest to the tip of the tracked instrument, thus presenting to the surgeon an OCT representation of the tissue that is closest to the surgical instrument, and which is thus of most immediate interest to the surgeon (see FIG. 6). Additionally, by identifying the instrument (e.g., membrane peeler) that is actually being used, the system can change the parameters that are being displayed in the HUD to the surgeon, including any warnings. For example, and with reference to FIG. 7, when the instrument being used is a hook, the unique indicia, marking, or otherwise on the instrument, and particularly advantageously at or immediately adjacent to the tip of instrument so it is most likely to be within the field of view of the optical system and the surgeon, is identified by the system, which then modifies the images presented in the HUD to include a distance meter or the like, and removes other parameter data from the HUD that is no longer relevant, because the instrument(s) from which that data is derived is no longer being used. By way of a non-limiting example, the system includes a database, e.g., a simple look-up table, in which each instrument's identity is correlated with its unique indicia, marking, etc. When the system identifies the indicia on or in the instrument being used, the system looks up the corresponding instrument. In the same or another database to which the system as access, the particular instrument is associated with one or more parameters related to the use of that instrument. The system then automatically generates images in the HUD of these real-time parameter data, as described elsewhere herein (see, e.g., FIGS. 1, 2, and 4), optionally permitting the surgeon or another person to select from among the several possible parameters to display. In another example, when the instrument is a cutting instrument, the system automatically identifies the instrument, and changes the parameters displayed in the HUD to include those for the cutting instrument, e.g., speed. The same methodology applies to any instrument used in a vitrectomy procedure. As different instruments are introduced into the surgical field, the system automatically identifies the new instrument, and adapts the display to include parameter data that is relevant to that instrument, and advantageously removes displays of parameter data that is no longer relevant because the instrument to which it relates is no longer being used.

By way on non-limiting examples, one or more of the standard instruments can be marked with nanoparticles, infrared coatings, dyes, etchings, grooves, patterns, or the like, which can be detected by the system analyzing image data of the surgical field, and different instruments have different markings, thus enabling the system to determine which instrument is actually being used in a surgery in real time. More specifically, the real-time image data of the instrument passes through the microscope and to the system, which uses standard optical and/or character recognition subsystems to recognize the instrument's external marking(s) and thus locate the exact location of the marking, and thus the location and identity of the instrument, in the ocular globe. Thus, for example, if an infrared coating system is used on a set of instruments, the optical system is sensitive in the infrared range and thus can detect these coatings and identify the instrument, and so forth. This location data is then used in one or more ways by the system, including presenting the distance numerically in the HUD overlay to the surgeon, and/or graphically (e.g., a bar of changing length). Furthermore, the location data can also optionally be used to modify the 3D representation of the OCT data, and more specifically, the distance of the 3D representation of the instrument's tip relative to the uppermost tissue layer of the posterior segment of the ocular globe. By way of a non-limiting example, a set of instruments, e.g., vitrectomy instruments as discussed herein, includes at least two different types of instruments, and each type of instrument has different indicia from the other instrument type(s), and all of the indicia are recognizable by the system. In this way, the system can identify the type of instrument that is present in the surgical field, and thus differentiate between the at least two different types of instruments. Based on that indicia-based identification, the system can perform additional tasks as described herein.

Further optionally, and with reference to FIG. 8, one or more of the vitrectomy instruments I can be modified to include a light tube 30, e.g., fiber optic waveguide, which can be used to collect data representative of the distance of the instrument from adjacent tissue, which can be used in any of the methods described above. Other embodiments include the capacity to perform real-time OCT data collection from the instrument itself, by the inclusion of a suitable light source 32 (e.g., superluminescent diodes, ultrashort pulsed lasers, supercontinuum lasers) at or near the working end of the vitrectomy instrument I, with the data communicated to the system, which can therefore also generate data representative of the distance from the instrument to the tissue. FIG. 8 also illustrates exemplary indicia 40, here represented by different numbers of lines and/or dots, different sizes of lines and/or dots, numbers and/or letters, or combinations thereof, including in different orders, orientations, and/or patterns. Thus, a set of instruments would have different indicia for each type of instrument, size of instrument, etc., and thus can be uniquely identified by the system.

The data manipulations described herein will be readily understood by those of ordinary skill in the art. According to a preferred embodiment, with reference to FIG. 9, a system as described herein performs the data manipulations by a set of executable logic instructions (e.g., a software program) implemented on a processing unit having access to a memory. The set of instruction may be stored in non-transitory computer-readable media in or coupled to a processing unit in the form of computer instructions to be executed by the processing unit for carrying out the image processing techniques disclosed herein. By way of example, the program includes an input module, an output module, an overlay module, a registration module, a motion tracking module, and a rendering module, such as those described in the aforementioned patent documents. Image data is obtained by a device or devices 40 which are commonly used in posterior segment visualization and surgery, such as those described in the documents cited herein, and their outputs are input to the system's input/output. Thus, the system includes an optical data gathering system, and an instrument-identification system in communication with the optical data gathering system, the instrument-identification system including data representative of the external surface indicia and data representative of a type of vitrectomy instrument associated with said external surface indicia. The system thus also includes an instrument-tracking system in data communication with an optical data gathering system, and a processing system that receives data from the instrument-tracking system indicative of a location of a tip of the instrument, directs the OCT system to gather OCT data concerning tissue closest to that instrument tip, and to direct the display system to display that OCT data. As discussed above, it can be particularly advantageous when that OCT data is displayed immediately adjacent to the location of the instrument tip, either the actual view of the tip that the surgeon has through the surgical microscope, or a representation of the tip of the instrument generated by the display unit. Furthermore, as discussed elsewhere herein, the system thus also includes subsystems, modules, or the like so that the OCT system gathers 2D OCT data in (at least) two orthogonal planes, and the display system displays that 2D OCT data intersecting at a point immediately adjacent to the instrument tip (or its representation, as discussed above).

While the invention has been described in detail with reference to exemplary embodiments thereof, it will be apparent to one skilled in the art that various changes can be made, and equivalents employed, without departing from the scope of the invention. The foregoing description of the preferred embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

I claim:

1. A system useful in posterior segment visualization and surgery, the system comprising:
at least two vitrectomy instruments, each instrument including external surface indicia that is different from the other of said at least two vitrectomy instruments;
an optical data gathering system configured and arranged for use during posterior segment visualization and surgery;
an instrument-identification system in communication with the optical data gathering system, the instrument-identification system including data representative of said external surface indicia and data representative of a type of vitrectomy instrument associated with said external surface indicia.

2. A system according to claim 1, further comprising:
a display system in communication with said instrument-identification system, said display system configured and arranged to display parameter data based on said data representative of a type of vitrectomy instrument associated with said external surface indicia.

3. A system according to claim 2, further comprising:
wherein said display system is further configured and arranged to not display parameter data not associated with said data representative of a type of vitrectomy instrument associated with said external surface indicia.

4. A system according to claim 2, wherein said display is a heads-up display.

5. A system according to claim 1, wherein said external surface indicia are visually detectable.

6. A system according to claim 1, wherein said external surface indicia are at or adjacent to a distal tip of each instrument.

7. A system useful in posterior segment visualization and surgery, the system comprising:
an optical data gathering system configured and arranged for use during posterior segment visualization and surgery;
an instrument-tracking system in communication with said optical data gathering system;
an optical computer tomography (OCT) data gathering system configured for use during posterior segment visualization and surgery;
a display system configured and arranged to display images representative of data from said said OCT system; and
a processing system configured and arranged to receive data from said instrument-tracking system indicative of a location of a tip of a vitrectomy instrument, to direct said OCT system to gather OCT data concerning tissue closest to said instrument tip, and to direct said display system to display said OCT data.

8. A system according to claim 7, wherein said display system is configured and arranged to display said OCT data immediately adjacent to the location of said instrument tip.

9. A system according to claim 8, wherein:
said OCT system is configured and arranged to gather 2D OCT data in two orthogonal planes; and
said display system is configured and arranged to display said 2D OCT data intersecting at a point immediately adjacent to said instrument tip.

10. A method of operating a posterior segment visualization and surgery system, the method comprising:
(a) locating the tip of a vitrectomy instrument with an optical data gathering system;
(b) gathering data with an optical computer tomography (OCT) data gathering system concerning tissue closest to said instrument tip;
(c) displaying an image representative of said OCT data;
moving said instrument tip to a new location; and
repeating steps (a), (b), and (c) for said new location of said instrument tip.

11. A method according to claim 10, wherein displaying an image further comprises displaying an image representative of the instrument tip.

12. A method according to claim 10, further comprising:
identifying external surface indicia on said instrument with said optical data gathering system; and
displaying an image representative of parameter data of the use of the instrument.
